(19) United States
(12) Patent Application Publication
(10) Pub. No.: US 2006/0052646 A1
(43) Pub. Date: Mar. 9, 2006

(54) Process for producing ether compounds in presence of a copper (II) salt

(76) Inventors: Wataru Oguchi, Oita (JP); Hiroshi Uchida, Oita (JP)

Correspondence Address: SUGHRUE MION, PLLC, 2100 PENNSYLVANIA AVENUE, N.W., SUITE 800, WASHINGTON, DC, 20037, US

(21) Appl. No.: 10/517,373

(22) PCT Filed: May 28, 2003
(86) PCT No.: PCT/JP03/06710

§ 371 Date: Jul. 28, 2005

Related U.S. Application Data
(60) Provisional application No. 60/388,735, filed on Jun. 17, 2002.

(30) Foreign Application Priority Data
Jun. 12, 2002 (JP) 2002-170988

(51) Int. Cl. C07C 41/06

(52) U.S. Cl. 568/626; 568/689

(57) ABSTRACT

A process for producing allylic ether compounds in presence of a catalyst comprising at least one Cu(II) salt by reaction of an allylic alcohol with either itself or another alcohol.

This application claims the priority of an application based on U.S. Provisional Application Ser. No. 60/388,735 (filed on Jun. 17, 2002).

### TECHNICAL FIELD

The present invention relates to a catalyst comprising a divalent copper compound, which is usable for an etherification reaction, a process for producing an ether compound by using the catalyst, and an ether compound obtained by the such a production process.

More specifically, the present invention relates to a catalyst comprising a divalent copper compound, which is usable in an etherification reaction between an alcohol compound and a compound having at least one hydroxyl group within the molecule thereof, or in an etherification reaction between an ether compound and a compound having at least one hydroxyl group within the molecule; a process for producing an ether compound, comprising conducting an etherification reaction of an alcohol compound or an etherification reaction of an ether compound and a compound having at least one hydroxyl group within the molecule thereof, in the presence of the catalyst; and an ether compound produced by such a production process.

### BACKGROUND ART

Allyl ethers are known as a useful organic intermediate compound to be used for reaction diluents, unsaturated monomers, epoxy resin production and the like. The process (called “Williamson reaction”) for producing an ether compound from an alkyl halide and a metal alkoxide is a most common process for producing an ether compound. In practice, as an example of the synthesis of an unsaturated ether compound, it is well known to produce an ethyl vinyl ether which is an unsaturated ether compound, from allyl chloride and sodium ethoxide.

However, this process is not practical as an industrial process for producing an unsaturated ether compound, because the metal alkoxide must be separately synthesized in advance from an alcohol compound and metal sodium, and further, a large amount of salt is inevitably produced as a by-product.

Another well-known reaction corresponds to a process for producing an ether compound by a dehydration reaction of an alcohol compound using, as the dehydration catalyst, an acid catalyst such as sulfuric acid, hydrochloric acid, aromatic sulfonic acid, sulfonic acid chloride, boron trifluoride and aluminum chloride. For example, S. Sugasawa, K. Fujiwara, et al., Organic Synthesis, IV, 72 (1963), report that a corresponding 2-chloroethyl(α-phenylbenzyl) ether is obtained from α-phenylbenzene methanol and 2-chloroethanol in the presence of sulfuric acid. However, in the process for producing an unsaturated ether compound by the dehydration reaction of an alcohol compound, a high reactivity is attained only in a case where the alcohol compound used for such a purpose has a tertiary alkyl group or a benzyl group, but a sufficiently high yield cannot be obtained in a case where another alcohol compound is used. Accordingly, in this method, the alcohol compounds which are usable for this purpose are rather limited.

On the other hand, Tetsuya Ogura, Nobuo Furuno and Shinichi Kawaguchi, Bulletin of the Chemical Society of Japan, Vol. 42, 643 (1969) report a process for selectively producing a diallyl ether by an etherification reaction of an allyl alcohol using a catalyst comprising copper(I) (or cuprous) chloride and ammonium chloride. However, in this reaction, the co-presence of the ammonium chloride the amount of which is almost equivalent to that of the copper(I) chloride is necessary for the purpose of obtaining a sufficiently high reaction rate. Accordingly, the industrial applicability of this method is considerably problematic.

As described above, the processes for producing various kinds of ether compounds, particularly allyl ethers, have been proposed in various ways. However, an industrially applicable production method therefor has not yet been reported.

### DISCLOSURE OF INVENTION

An object of the present invention is to provide a catalyst for producing an ether compound, which has solved the above-mentioned problems encounterd in the prior art.

Another object of the present invention is to provide a catalyst for producing an ether compound, which is usable in an etherification reaction between an alcohol compound and a compound having at least one hydroxyl group within the molecule thereof, or in an etherification reaction between an ether compound and a compound having at least one hydroxyl group within the molecule thereof.

A further object of the present invention is to provide a process for producing an ether compound using such a catalyst; and an ether compound which is obtainable by this production process.

As a result of earnest study, the present inventors have found that a catalyst comprising divalent copper(II) effectively acts as the catalyst for the etherification reaction between an alcohol compound and a compound having at least one hydroxyl group within the molecule thereof or for the etherification reaction between an ether compound and a compound having at least one hydroxyl group within the molecule thereof, to thereby provide an intended ether compound with high selectivity. The present invention has been accomplished based on such a discovery.

More specifically, the present invention (I) relates to a catalyst for producing an ether compound, comprising at least one copper compound selected from the group consisting of: copper(II) sulfate, copper(II) ammonium chloride, copper(II) carbonate, copper(II) diphosphate, copper(II) formate, copper(II) gluconate, copper(II) hydroxide, copper(II) nitrate, copper(II) oleate, copper(II) oxalate, copper(II) sulfide, copper(II) phthalate, copper(II) phthalocyanine, copper(II) potassium chloride, copper(II) terephthalate, copper(II) thiocyanate, copper(II) chloride, copper(II) bromide, copper(II) fluoride, copper(II) iodide, copper(II) oxide, copper(II) acetate, bis(acetylacetonato)-copper(II) and hydrates of these copper(II) compounds.

The present invention (II) relates to the catalyst for producing an ether compound according to the present invention (I), which is a catalyst usable in the production of an ether compound represented by the general formula (3) from an alcohol compound represented by the general formula (1) and a compound represented by the general formula (2):
Formula (1): 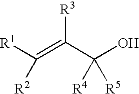
(wherein R¹, R², R³, R⁴ and R⁵ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R⁶—OH Formula (2):
(wherein R⁶ represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
Formula (3): 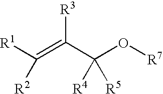
(wherein R¹, R², R³, R⁴, R⁵ and R⁷ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

The present invention (III) relates to the catalyst for producing an ether compound according to the present invention (I), which is a catalyst usable in the production of an ether compound represented by the general formula (6) from an ether compound represented by the general formula (4) and a compound represented by the general formula (5):
R⁸—O—R⁹ Formula (4):
(wherein R⁸ and R⁹ each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹⁰—OH Formula (5):
(wherein R¹⁰ represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹¹—O—R¹² Formula (6):
(wherein R¹¹ and R¹² each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

The present invention (IV) relates to a process for producing an ether compound represented by the general formula (3), comprising conducting an etherification reaction between a compound represented by the general formula (1) and a compound represented by the general formula (2) in the presence of the catalyst for producing an ether compound according to the present invention (II):
Formula (1): 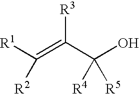
(wherein R¹, R², R³, R⁴ and R⁵ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R⁶—OH Formula (2):
(wherein R⁶ represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
Formula (3): 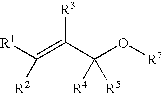
(wherein R¹, R², R³, R⁴, R⁵ and R⁷ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

The present invention (V) relates to a process for producing an ether compound represented by the general formula (6), comprising conducting an etherification reaction between a compound represented by the general formula (4) and a compound represented by the general formula (5) in the presence of the catalyst for producing an ether compound according to the present invention (III):
R⁸—O—R⁹ Formula (4):
(wherein R⁸ and R⁹ each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹⁰—OH Formula (5):
(wherein R¹⁰ represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹¹—O—R¹² Formula (6):
(wherein R¹¹ and R¹² each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

The present invention (VI) relates to an ether compound produced by the process for producing an ether compound according to the present invention (IV) or (V).

For example, the present invention includes the following embodiments.

[1] A catalyst for producing an ether compound, comprising at least one copper compound selected from the group consisting of: copper(II) sulfate, copper(II) ammonium chloride, copper(II) carbonate, copper(II) diphosphate, copper(II) formate, copper(II) gluconate, copper(II) hydroxide, copper(II) nitrate, copper(II) oleate, copper(II) oxalate, copper(II) sulfide, copper(II) phthalate, copper(II) phthalocyanine, copper(II) potassium chloride, copper(II) terephthalate, copper(II) thiocyanate, copper(II) chloride, copper(II) bromide, copper(II) fluoride, copper(II) iodide, copper(II) oxide, copper(II) acetate, bis(acetylacetonato)-copper(II) and hydrates thereof.

[2] The catalyst for producing an ether compound according to [1], which is a catalyst usable in the production of an ether compound represented by the general formula (3) from an alcohol compound represented by the general formula (1) and a compound represented by the general formula (2):
Formula (1): 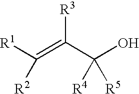
(wherein R¹, R², R³, R⁴ and R⁵ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R⁶—OH Formula (2):
(wherein R⁶ represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
Formula (3): 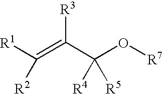
(wherein R¹, R², R³, R⁴, R⁵ and R⁷ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

[3] The catalyst for producing an ether compound according to [1], which is a catalyst usable in the production of an ether compound represented by the general formula (6) from an ether compound represented by the general formula (4) and a compound represented by the general formula (5):
R⁸—O—R⁹ Formula (4):
(wherein R⁸ and R⁹ each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹⁰—OH Formula (5):
(wherein R¹⁰ represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹¹—O—R¹² Formula (6):
(wherein R¹¹ and R¹² each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

[4] A process for producing an ether compound represented by the general formula (3), comprising conducting an etherification reaction between a compound represented by the general formula (1) and a compound represented by the general formula (2) in the presence of the catalyst for producing an ether compound according to [2]:
Formula (1): 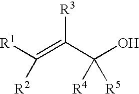
(wherein R¹, R², R³, R⁴ and R⁵ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R⁶—OH Formula (2):
(wherein R⁶ represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
Formula (3): 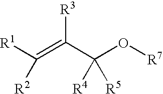
(wherein R¹, R², R³, R⁴, R⁵ and R⁷ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

[5] The process for producing an ether compound according to [4], wherein the alcohol compound represented by the general formula (1) is a compound having from 2 to 20 carbon atoms.

[6] The process for producing an ether compound according to [4] or [5], wherein the alcohol compound represented by the general formula (1) is at least one compound selected from the group consisting of: allyl alcohol, 2-methyl-2-propen-1-ol, 3-buten-2-ol, 2,3-dimethyl-3-buten-2-ol, 3-methyl-3-buten-2-ol, 2-buten-1-ol, 2-methyl-3-buten-1-ol and 3-penten-2-ol.

[7] The process for producing an ether compound according to any one of [4] to [6], wherein the compound represented by the general formula (2) is at least one compound selected from the group consisting of: alcohol compounds, phenol compounds, polycondensation reaction products from a phenol compound and an aldehyde compound, and polyaddition reaction products from a phenol compound and an unsaturated hydrocarbon compound.

[8] The process for producing an ether compound according to [7], wherein the alcohol compound is at least one compound selected from the group consisting of: vinyl alcohol, 2-methylvinyl alcohol, allyl alcohol, 2-methyl-2-propen-1-ol, 3-buten-2-ol, 2,3-dimethyl-3-buten-2-ol, 3-methyl-3-buten-2-ol, 2-buten-1-ol, 2-methyl-3-buten-1-ol, 3-penten-2-ol, ethylene glycol, ethylene glycol mono-substituted product, 1,2-propanediol, 1,2-propanediol mono-substituted product, 1,3-propanediol, 1,3-propanediol mono-substituted product, 1,2-butanediol, 1,2-butanediol mono-substituted product, 1,3-butanediol, 1,3-butanediol mono-substituted product, 1,4-butanediol, 1,4-butanediol mono-substituted product, trimethylol propane, trimethylol propane mono-substituted product, trimethylol propane di-substitution product, pentaerythritol, pentaerythritol mono-substituted product, pentaerythritol di-substitution product, pentaerythritol tri-substituted product, methanol, ethanol, 1-propanol, 2-propanol, 1-butanol, 2-butanol, tert-butanol and benzyl alcohol.

[9] The process for producing an ether compound according to [7] or [8], wherein the phenol compound is at least one compound selected from the group consisting of: unsubstituted phenol, mono-substituted phenol compounds, di-substituted phenol compounds, tri-substituted phenol compounds, dihydric phenol compounds and naphthol compounds.

[10] The process for producing an ether compound according to any one of [4] to [9], wherein the alcohol compound represented by the general formula (1) is allyl alcohol.

[11] A process for producing an ether compound represented by the general formula (6), comprising conducting an etherification reaction between a compound represented by the general formula (4) and a compound represented by the general formula (5) in the presence of the catalyst for producing an ether compound according to [3]:
R⁸—O—R⁹ Formula (4):
(wherein R⁸ and R⁹ each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹⁰—OH Formula (5):
(wherein R¹⁰ represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹¹—O—R¹² Formula (6):
(wherein R¹¹ and R¹² each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

[12] The process for producing an ether compound according to [11], wherein the compound represented by the general formula (4) is at least one compound selected from the group consisting of: allyl ether compounds, vinyl ether compounds and propenyl ether compounds and the compound represented by the general formula (5) is at least one compound selected from the group consisting of: alcohol compounds, phenol compounds, polycondensation reaction products from a phenol compounds and an aldehyde compound, and polyaddition reaction products from a phenol compound and an unsaturated hydrocarbon compound.

[13] The process for producing an ether compound according to [11] or [12], wherein the ether compound represented by the general formula (4) is diallyl ether.

[14] The process for producing an ether compound according to any one of [4] to [13], wherein water is present in the reaction system.

[15] The process for producing an ether compound according to [14], wherein the amount of water present in the reaction system is from 0.01 to 40% by mass.

[16] An ether compound produced by the process for producing an ether compound according to any one of [4] to [15].

### BEST MODE FOR CARRYING OUT THE INVENTION

Hereinbelow, the present invention will be described in detail. In the following description, “%” and “part(s)” representing a quantitative proportion or ratio are those based on mass, unless otherwise noted specifically.

(Present Invention (I))

The present invention is described in detail below. The present invention (I) is first described.

The present invention (I) is a catalyst for producing an ether compound, comprising at least one copper compound selected from the group consisting of: copper(II) sulfate, copper(II) ammonium chloride, copper(II) carbonate, copper(II) diphosphate, copper(II) formate, copper(II) gluconate, copper(II) hydroxide, copper(II) nitrate, copper(II) oleate, copper(II) oxalate, copper(II) sulfide, copper(II) phthalate, copper(II) phthalocyanine, copper(II) potassium chloride, copper(II) terephthalate, copper(II) thiocyanate, copper(II) chloride, copper(II) bromide, copper(II) fluoride, copper(II) iodide, copper(II) oxide, copper(II) acetate, bis(acetylacetonato)-copper(II), and hydrates of these compounds.

The divalent copper compound to be used in the catalyst for producing an ether compound according to the present invention (I) is not particularly limited, and may take any form or shape as long as the valence number of the copper atom in the copper compound is divalent. Of course, the divalent copper compound may be changed so as to have a different valence number in the transition state during the reaction.

The divalent copper compound may preferably be at least one member selected from the group consisting of: copper(II) sulfate, copper(II) ammonium chloride, copper(II) carbonate, copper(II) diphosphate, copper(II) formate, copper(II) gluconate, copper(II) hydroxide, copper(II) nitrate, copper(II) oleate, copper(II) oxalate, copper(II) sulfide, copper(II) phthalate, copper(II) phthalocyanine, copper(II) potassium chloride, copper(II) terephthalate, copper(II) thiocyanate, copper(II) chloride, copper(II) bromide, copper(II) fluoride, copper(II) iodide, copper(II) oxide, copper(II) acetate, bis(acetylacetonato)-copper(II), and hydrates of these compounds. Among these divalent copper compounds, more preferred are copper(II) chloride and copper(II) chloride dihydrate.

In the present invention (I), the divalent copper compound can be used in any form or shape and the form or shape is not particularly limited. The divalent copper compound may be used in the form of powder, pellet, or in a form such that the divalent copper compound is supported on a carrier (or support). It is easiest that the divalent copper compound is in the form of powder, or is supported on a carrier. Preferred examples of the carrier may include: silica, alumina, magnesia, activated carbon, silica-alumina, titania and zirconia, but the present invention is not limited to these specific examples.

The method of causing the divalent copper compound to be supported on a carrier is not particularly limited, it is possible to use a known method such as impregnation, spraying, evaporation to dryness, kneading and spray-drying. The divalent copper compound may contain other metal element(s), a copper compound having a different valence number and an organic compound. In view of the reduction of by-product(s), the purity of the divalent copper compound or divalent copper compound hydrate may preferably be 50% by mass or more, more preferably 70% by mass or more, more preferably 80% by mass or more.

(Present Invention (II) and (III))

The present invention (II) and the present invention (III) are described below.

The present invention (II) is the catalyst for producing an ether compound according to the present invention (I), which is a catalyst usable in the production of an ether compound represented by the general formula (3) from an alcohol compound represented by the general formula (1) and a compound represented by the general formula (2):
Formula (1): 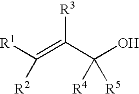
(wherein R¹, R², R³, R⁴ and R⁵ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R⁶—OH Formula (2):
(wherein R⁶ represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
Formula (3): 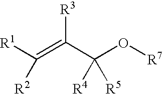
(wherein R¹, R², R³, R⁴, R⁵ and R⁷ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

The present invention (III) is the catalyst for producing an ether compound according to the present invention (I), which is a catalyst usable in the production of an ether compound represented by the general formula (6) from an ether compound represented by the general formula (4) and a compound represented by the general formula (5):
R⁸—O—R⁹ Formula (4):
(wherein R⁸ and R⁹ each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹⁰—OH Formula (5):
(wherein R¹⁰ represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹¹—O—R¹² Formula (6):
(wherein R¹¹ and R¹² each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

The catalyst for producing an ether compound according to the present invention (II) or the present invention (III) is a catalyst comprising a divalent copper compound and is effective for an etherification reaction between an alcohol compound and a compound having at least one hydroxyl group within the molecule thereof, or for an etherification reaction between an ether compound and a compound having at least one hydroxyl group within the molecule thereof.

(Present Invention (IV))

The present invention (IV) is described below.

The present invention (IV) is a process for producing an ether compound represented by the general formula (3), comprising conducting an etherification reaction between a compound represented by the general formula (1) and a compound represented by the general formula (2) in the presence of the catalyst for producing an ether compound according to the present invention (II):
Formula (1): 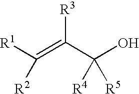
(wherein R¹, R², R³, R⁴ and R⁵ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R⁶—OH Formula (2):
(wherein R⁶ represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
Formula (3): 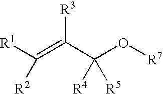
(wherein R¹, R², R³, R⁴, R⁵ and R⁷ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

According to the process for producing an ether compound according to the present invention (IV), an ether compound represented by the general formula (3) can be produced from a compound represented by the general formula (1) and a compound represented by the general formula (2) in a high yield with high selectivity. The compound represented by the general formula (1) and the compound represented by the general formula (2), which are usable in the present invention (IV), are not particularly limited. Of course, the compound represented by the general formula (1) and the compound represented by the general formula (2) may be the same. Further, the ether compound obtained by the etherification reaction between the compound represented by the general formula (1) and the compound represented by the general formula (2) in the presence of the catalyst comprising a divalent copper compound of the present invention (IV) is not limited to one kind thereof, and the present invention includes a case where several kinds of ether compounds are obtained.

For example, the present invention (IV) includes a case such that in an etherification reaction between the compound represented by the general formula (1) and the compound represented by the general formula (2), a monoether compound having one ether bond and a diether compound having two ether bonds are simultaneously obtained. More specifically, 1,3-propanediol monoallyl ether and 1,3-propanediol diallyl ether are simultaneously obtained by an etherification reaction between allyl alcohol and 1,3-propanediol in the presence of the catalyst for producing an ether compound according to the present invention (II) and, needless to say, this case is included in the present invention (IV).

Further, the present invention (IV) includes, for example, a case such that in an etherification reaction between the compound represented by the general formula (1) and the compound represented by the general formula (2), a symmetric ether compound is obtained from two molecules of the compound represented by the general formula (1) and an asymmetric ether compound is simultaneously obtained from the compound represented by the general formula (1) and the compound represented by the general formula (2). More specifically, the present invention (IV) includes a case where diallyl ether and diethyl ether as symmetric compounds and allyl ethyl ether as an asymmetric ether compound are obtained by an etherification reaction between diallyl ether and ethanol in the presence of the catalyst for producing an ether compound according to the present invention (II).

The compound represented by the general formula (1) to be used in the present invention (IV) may preferably be an alcohol compound having from 2 to 20 carbon atoms. Preferred examples of the alcohol compound may include: allyl alcohol, 2-methyl-2-propen-1-ol, 3-buten-2-ol, 2,3-dimethyl-3-buten-2-ol, 3-methyl-3-buten-2-ol, 2-buten-1-ol, 2-methyl-3-buten-1-ol and 3-penten-2-ol. Among these, more preferred is ally alcohol.

On the other hand, preferred examples of the compound represented by the general formula (2) to be used in the present invention (IV) may include: alcohol compounds, phenol compounds, polycondensation reaction products from a phenol compound and an aldehyde compound, and polyaddition reaction products from a phenol compound and an unsaturated hydrocarbon compound. More specifically, examples of the alcohol compounds may include: vinyl alcohol, 2-methylvinyl alcohol, allyl alcohol, 2-methyl-2-propen-1-ol, 3-buten-2-ol, 2,3-dimethyl-3-buten-2-ol, 3-methyl-3-buten-2-ol, 2-buten-1-ol, 2-methyl-3-buten-1-ol, 3-penten-2-ol, ethylene glycol, ethylene glycol mono-substituted product, 1,2-propanediol, 1,2-propanediol mono-substituted product, 1,3-propanediol, 1,3-propanediol mono-substituted product, 1,2-butanediol, 1,2-butanediol mono-substituted product, 1,3-butanediol, 1,3-butanediol mono-substituted product, 1,4-butanediol, 1,4-butanediol mono-substituted product, trimethylol propane, trimethylol propane mono-substituted product, trimethylol propane di-substitution product, pentaerythritol, pentaerythritol mono-substituted product, pentaerythritol di-substitution product, pentaerythritol tri-substituted product, methanol, ethanol, 1-propanol, 2-propanol, 1-butanol, 2-butanol, tert-butanol and benzyl alcohol.

The phenol compound may preferably be at least one compound selected from the group consisting of: unsubstituted phenol, mono-substituted phenol compounds, di-substituted phenol compounds, tri-substituted phenol compounds, dihydric phenol compounds, naphthol compounds, polycondensation reaction products from such a phenol compound and an aldehyde compound, and polyaddition reaction products from such a phenol compound and an unsaturated hydrocarbon compound.

More specifically, examples of the mono-substituted phenol compounds may include: cresol, xylenol, ethylphenol, isopropylphenol, butylphenol, octylphenol, nonylphenol, vinylphenol, isopropenylphenol, allylphenol, phenylphenol, benzylphenol, chlorophenol and bromophenol (each of the above phenol compounds including ortho-, meta- and para-isomers). Examples of the di-substituted phenol compounds may include: dimethylphenol, diethylphenol and tert-butyl-methylphenol (each of the above phenol compounds including isomers). Examples of the tri-substituted phenols may include: trimethylphenol. Examples of the dihydric phenol compounds may include: bisphenol A, bisphenol F, bisphenol AD, bisphenol S, dihydroxybenzophenone, hydroquinone, resorcin, dihydroxynaphthalene, biphenol and binaphthol. Examples of the naphthol compounds may include: 1-naphthol, 2-naphthol and dihydroxynaphthalene. Examples of the polycondensation reaction product of a phenol compound and an aldehyde compound may include: a polycondensation reaction product of a phenol compound and formaldehyde, a polycondensation reaction product of a phenol compound and acetaldehyde, and a polycondensation reaction product of a phenol compound and hydroxybenzaldehyde. Examples of the polyaddition reaction product of a phenol compound and an unsaturated hydrocarbon compound may include: a polyaddition reaction product of a phenol compound and dicyclopentadiene, a polyaddition reaction product of a phenol compound and tetrahydroindene, a polyaddition reaction product of a phenol compound and 4-vinylcyclohexene, a polyaddition reaction product of a phenol compound and 5-vinylnorbon-2-ene, a polyaddition reaction product of a phenol compound and α-pinene, a polyaddition reaction product of a phenol compound and β-pinene, a polyaddition reaction product of a phenol compound and limonene, and a polyaddition reaction product of a phenol compound and divinylbenzene.

The etherification reaction according to the present invention (IV) can be conducted in any phase state of gas phase, liquid phase or solid phase. In some cases, different phase states may be present in a combination with each other. The reaction system and the form or type of the reaction apparatus to be used in the etherification reaction are not particularly limited The reaction may be conducted in a batch system, a continuous system or a semi-continuous system, by using an appropriate reaction apparatus such as fixed-bed reaction apparatus, moving-bed reaction apparatus, fluidized-bed reaction apparatus, tank-type reaction apparatus, reaction distillation apparatus or continuous stirring tank-type reaction apparatus. Any of these methods may be used. Preferred reaction apparatuses are a fixed-bed reaction apparatus, a fluidized-bed reaction apparatus, a tank-type reaction apparatus and a reaction distillation apparatus, and preferred reaction systems are a batch system and a continuous system.

In the etherification reaction according to the present invention (IV), the compound represented by the general formula (1), the compound represented by the general formula (2) and the catalyst comprising a divalent copper compound can be used in an arbitrary ratio without particular limitation. In addition to these components, different component(s) may be present in the reaction system and the presence of another component causes substantially no trouble.

In the etherification reaction, the preferred amount of the catalyst comprising a divalent copper compound may somewhat vary depending on the reaction system, the reactivity of the compound represented by the general formula (1), the reactivity of the compound represented by the general formula (2), the activity of the catalyst, and the reaction conditions therefor. For example, in a liquid phase reaction using a tank-type reaction apparatus, the amount of the catalyst comprising a divalent copper compound is, in terms of the divalent copper compound, from 0.001 to 10.0 mol %, more preferably from 0.01 to 5.0 mol %, still more preferably from 0.02 to 0.1 mol %, based on the compound represented by the general formula (1). In a case where a fixed-bed reaction apparatus or a fluidized-bed reaction apparatus is used, the amount may apparently be larger than this range.

In the etherification reaction according to the present invention (IV), the preferred ratio of the compound represented by the general formula (1) to be charged into the reaction system, to the compound represented by the general formula (2) may somewhat vary depending on the molecular structure and reactivity of the compound represented by the general formula (1) and the compound represented by the general formula (2), and the kind of an intended ether compound.

For example, in the etherification reaction between the compound represented by the general formula (1) and the compound represented by the formula (2), the ratio of the compound represented by the general formula (1) may preferably be from 50 to 600 mol %, more preferably from 80 to 300 mol %, still more preferably from 100 to 250 mol %, based on the compound represented by the general formula (2).

In the etherification reaction according to the present invention (IV), the method of mixing the compound represented by the general formula (1), the compound represented by the general formula (2) and the catalyst comprising a divalent copper compound is not particularly limited and these components may be mixed by any of mixing methods. Specific examples of the method of mixing the compound represented by the general formula (1), the compound represented by the general formula (2) and the catalyst comprising a divalent copper compound may include: in the case of a flow-type reaction using a fixed-bed reaction apparatus, a method of passing the compound represented by the general formula (1) and the compound represented by the general formula (2) each in a gas or liquid state through the catalyst comprising a divalent copper compound fixed in a reactor to thereby conduct the etherification reaction. In some cases, the etherification reaction may also be conducted by passing the compound represented by the general formula (1) and the compound represented by the general formula (2), while these compounds are brought into countercurrent contact with each other in a reactor.

In the case of a batch-system reaction using a tank-type reaction apparatus, examples of the mixing method may include: a method of previously allowing the catalyst comprising a divalent copper compound to be present in a reactor, then adding to the reactor the compound represented by the general formula (1) and the compound represented by the general formula (2), and stirring the resultant mixture, to thereby conduct the etherification reaction. Of course, the present invention is not limited to these specific examples, and any of combinations of reactors, reaction processes, reaction system, the order of the components to be charged for the reaction, etc., can be used. For example, in the case of a batch-system reaction using a tank-type reaction apparatus, the etherification reaction can be also conducted by previously allowing the catalyst comprising a divalent copper compound to be present in a reactor, adding the compound represented by the general formula (1) to the reactor, stirring the resultant mixture for a while, then adding the compound represented by the general formula (2) to the reactor, and further stirring the resultant mixture.

(Separation of Catalyst)

The catalyst comprising a divalent copper compound and the reaction mixture can be separated by a conventionally known method. In the case of a flow-type reaction using a fixed-bed reaction apparatus, a reaction mixture free of a divalent copper compound is obtained directly from the reactor outlet and the catalyst and the reaction mixture can be easily separated.

On the other hand, in a case where the divalent copper compound is dissolved in the reaction mixture, it is possible that the reaction mixture containing the catalyst comprising a divalent copper compound is heated, whereby the compound represented by the general formula (1), the compound represented by the general formula (2) (which are raw materials) and an intended ether compound can be removed from the reaction mixture as evaporation components, and the catalyst comprising a divalent copper compound can be separated from the reaction mixture as a concentrated residue. The catalyst comprising a divalent copper compound which has been separated and recovered from the reaction mixture in this manner can be again used as the catalyst in the etherification reaction between the compound represented by the general formula (1) and the compound represented by the general formula (2). At this time, there arises substantially no problem, even if the recovered catalyst contains the compound represented by the general formula (1), the compound represented by the general formula (2), the ether compound, water as a by-product, and other high boiling point compounds.

(Reaction Temperature)

The reaction temperature in the etherification reaction according to the present invention (IV) is not particularly limited, and the etherification reaction may be conducted at any reaction temperature. The preferred reaction temperature may somewhat vary depending on the boiling point under atmospheric pressure of the compound represented by the general formula (1) to be used for the reaction. When the boiling point under atmospheric pressure of the compound represented by the general formula (1) may preferably be from 50 to 200° C., the reaction temperature may preferably be from 30 to 250° C., more preferably from 50 to 200° C. When the compound represented by the general formula (1) has a boiling point exceeding 200° C. under atmospheric pressure, the reaction temperature may preferably be from 80 to 300° C., more preferably from 100 to 250° C.

Of course, along with the change of reaction with the elapse of time, the etherification reaction can be conducted while changing the reaction temperature within the above-described range. If the reaction temperature is less than the preferred temperature, the etherification reaction may proceed at a low reaction rate and this is not practical. On the other hand, if the reaction temperature exceeds the preferred temperature, the amount of high-molecular weight impurities produced as by-products may be increased undesirably. In the etherification reaction according to the present invention (IV), the reaction pressure is not particularly limited and the etherification reaction may be conducted under any reaction pressure. The reaction pressure may be, for example, preferably from 0 to 4.0 MPaG (gauge pressure), more preferably from 0 to 3.0 MPaG.

In a case where the etherification reaction according to the present invention is conducted by using a closed-type reactor, the composition of constituent components contained in the reactor may be changed along with the progress of the reaction, and therefore, it is possible that the reaction pressure does not show a constant value and may take any value in a predetermined range. Even in such a case, an ether compound can be obtained without any problem.

In the etherification reaction according to the present invention (IV), when water is previously allowed to be present in the reaction system, an ether compound can be produced with high efficiency and high selectivity. Usually, in an etherification reaction by dehydration, the presence of water as a product is disadvantageous to the product side, in view of the equilibrium of reaction and is not preferred for efficiently obtaining an intended ether compound. However, in the reaction according to the present invention, the catalyst comprising a divalent copper compound is readily soluble in water. Accordingly, when water is present in the reaction system, the reaction rate may be increased.

Further, in the etherification reaction according to the present invention, when the product ether compound has a low solubility in water and the raw material compound represented by the general formula (1) has a high solubility in water, and water is present within the reaction system, a two-phase state is formed. One of the two phases is an organic phase which is rich in the produced ether compound. The other thereof is an aqueous phase which is rich in the catalyst comprising a divalent copper compound having a high solubility in water, and rich in the compound represented by the general formula (1). Therefore, the ether compound produced by an etherification reaction which has proceeded mainly in the aqueous phase is transferred from the aqueous phase to the organic phase side, and causes substantially no interaction with the catalyst comprising a divalent copper compound, which is present in the aqueous phase side, whereby an effect of suppressing the subsequent side reaction of the ether compound. Accordingly, in particular, when water is previously allowed to be present within the reaction system, an intended ether compound can be obtained with a remarkably high selectivity.

In the present invention, the concentration of water which is allowed to be present in the reaction system is not particularly limited. The water concentration may preferably be from 0.1 to 50% by mass, more preferably from 0.5 to 40% by mass, still more preferably from 1.0 to 30% by mass, based on the compound represented by the general formula (1) which is a raw material compound. In this case, the water previously allowed to be present in the reaction system of course includes water present as a hydrate (for example, hydrate of a divalent copper compound). The compound represented by the general formula (1), which is to be used in the process for producing an ether compound with high efficiency and high selectivity by previously allowing water to be present within the reaction system, is not particularly limited. Examples of the compound represented by the general formula (1) may include: alcohols having a high solubility in water, more specifically allyl alcohol.

In the present invention (IV), the crude product which has been obtained by separating the catalyst comprising a divalent copper compound from the mixture after reaction may be purified by a conventionally known method, whereby an ether compound having a high purity can be obtained. The method of such purification is not particularly limited. For example, an ether compound having a high purity can be obtained by subjecting a reaction mixture free of the catalyst comprising a divalent copper compound to at least one unit separating operation selected from the group consisting of: distillation, extraction, liquid-liquid separation, membrane separation and crystallization. Even if the catalyst comprising a divalent copper compound remains in the reaction mixture, an ether compound can be of course easily purified.

(Present Invention (V))

Next, the present invention (V) is described below.

The present invention (V) is a process for producing an ether compound represented by the general formula (6), comprising conducting an etherification reaction between a compound represented by the general formula (4) and a compound represented by the general formula (5) in the presence of the catalyst for producing an ether compound according to the present invention (III):
R⁸—O—R⁹ Formula (4):
(wherein R⁸ and R⁹ each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹⁰—OH Formula (5):
(wherein R¹⁰ represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹¹—O—R¹² Formula (6):
(wherein R¹¹ and R¹² each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

The compound represented by the general formula (4) and the compound represented by the general formula (5), which is usable in the present invention (V), are of course not limited by any means. The compound represented by the general formula (4) may preferably be at least one compound selected from the group consisting of: allyl ether compounds, vinyl ether compounds and propenyl ether compounds.

Specific examples thereof may include: methyl vinyl ether, ethyl vinyl ether, propyl vinyl ether, allyl methyl ether, allyl ethyl ether, allyl propyl ether, allyl isopropyl ether, allyl butyl ether, allyl pentyl ether, allyl isobutyl ether, diallyl ether, methyl-1-propenyl ether, ethyl-1-propenyl ether, propyl-1-propenyl ether, isopropyl-1-propenyl ether, butyl-1-propenyl ether, isobutyl-1-propenyl ether, ethylene glycol monovinyl ether, ethylene glycol divinyl ether, ethylene glycol monoallyl ether, ethylene glycol diallyl ether, ethylene glycol mono-1-propenyl ether, ethylene glycol di-1-propenyl ether, 1,2-propanediol monovinyl ether, 1,2-propanediol divinyl ether, 1,2-propanediol monoallyl ether, 1,2-propanediol diallyl ether, 1,2-propanediol mono-1-propenyl ether, 1,2-propanediol di-1-propenyl ether, 1,3-propanediol monovinyl ether, 1,3-propanediol divinyl ether, 1,3-propanediol monoallyl ether, 1,3-propanediol diallyl ether, 1,3-propanediol mono-1-propenyl ether, 1,3-propanediol di-1-propenyl ether, 1,2-butanediol monovinyl ether, 1,2-butanediol divinyl ether, 1,2-butanediol monoallyl ether, 1,2-butanediol diallyl ether, 1,2-butanediol mono-1-propenyl ether, 1,2-butanediol di-1-propenyl ether, 1,3-butanediol monovinyl ether, 1,3-butanediol divinyl ether, 1,3-butanediol monoallyl ether, 1,3-butanediol diallyl ether, 1,3-butanediol mono-1-propenyl ether, 1,3-butanediol di-1-propenyl ether, 1,4-butanediol monovinyl ether, 1,4-butanediol divinyl ether, 1,4-butanediol monoallyl ether, 1,4-butanediol diallyl ether, 1,4-butanediol mono-1-propenyl ether, 1,4-butanediol di-1-propenyl ether, trimethylolpropane monovinyl ether, trimethylolpropane divinyl ether, trimethylolpropane trivinyl ether, trimethylolpropane monoallyl ether, trimethylolpropane diallyl ether, trimethylolpropane triallyl ether, pentaerythritol monovinyl ether, pentaerythritol divinyl ether, pentaerythritol trivinyl ether, pentaerythritol tetravinyl ether, pentaerythritol monoallyl ether, pentaerythritol diallyl ether, pentaerythritol triallyl ether and pentaerythritol tetraallyl ether.

The preferred compound as the compound represented by the general formula (5) to be used in the present invention (V) is not particularly limited. Examples of the preferred compound as the compound represented by the general formula (5) to be used in the present invention (V) are the same as the preferred compound for the compound represented by the general formula (2) in the present invention (IV), and may include: for example, alcohol compounds, phenol compounds, polycondensation reaction products from a phenol compound and an aldehyde compound, and polyaddition reaction products from a phenol compound and an unsaturated hydrocarbon compound.

In the etherification reaction between the compound represented by the general formula (4) and the compound represented by the general formula (5) of the present invention (V), the ether compound to be obtained is of course not limited to one kind, and the present invention includes a case where two or more ether compounds are obtained at the same time. For example, 1,3-propanediol diallyl ether is obtained by an etherification reaction between 1,3-propanediol diethyl ether and ethylene glycol monoallyl ether, and this case is also of course included in the present invention (V).

The etherification reaction according to the present invention (V) can be conducted in the same manner as the present invention (IV). According to this etherification reaction, an ether compound different form the raw material can be produced from the compound represented by the general formula (4) and the compound represented by the general formula (5) with high efficiency and high selectivity. For example, the etherification reaction according to the present invention (V) can be conducted in any of phase states selected from a gas phase, a liquid phase or a solid phase. The reaction system and reaction apparatus form in the etherification reaction are not particularly limited. The reaction may be conducted in any of reaction systems including: batch system, continuous system or semi-continuous system, using an appropriate reaction apparatus such as fixed-bed reaction apparatus, moving-bed reaction apparatus, fluidized-bed reaction apparatus, tank-type reaction apparatus, reaction distillation apparatus or continuous stirring tank-type reaction apparatus. Preferred reaction apparatuses are a fixed-bed reaction apparatus, a fluidized-bed reaction apparatus, a tank-type reaction apparatus and a reaction distillation apparatus, and preferred reaction systems are a batch system and a continuous system.

In the etherification reaction according to the present invention (V), the compound represented by the general formula (4), the compound represented by the general formula (5), the ether compound and the catalyst comprising a divalent copper compound can be used in an arbitrary ratio without particular limitation. Components other than these components may also be present in the reaction system. The preferred amount of the catalyst comprising a divalent copper compound in the etherification reaction may somewhat vary depending on the reaction system, the reactivity of the compound represented by the general formula (4), the reactivity of the compound represented by the general formula (5), the activity of the catalyst, and the reaction conditions.

For example, in a liquid phase reaction using a tank-type reaction apparatus, the amount of the catalyst comprising a divalent copper compound may be, in terms of the divalent copper compound, from 0.001 to 10.0 mol %, more preferably from 0.01 to 5.0 mol %, still more preferably from 0.02 to 1.0 mol %, based on the compound represented by the general formula (4). In a case wherein a fixed-bed reaction apparatus or a fluidized-bed reaction apparatus is used, the amount of the catalyst may be apparently larger than this range. In the etherification reaction according to the present invention (V), the preferred ratio of the compound represented by the general formula (4) to the compound represented by the general formula (5) may somewhat vary depending on the reactivity of the compound represented by the general formula (4), the structure and reactivity of the compound represented by the general formula (5), and the kind of an intended ether compound. For example, in an etherification reaction between the compound represented by the general formula (4) and a compound having one hydroxyl group within the molecule thereof, the ratio of the compound represented by the general formula (4) may preferably be from 1.0 to 400 mol %, more preferably from 10 to 200 mol %, still more preferably from 20 to 150 mol %, based on the compound having one hydroxyl group within the molecule thereof. Further, for example, in an etherification reaction between the compound represented by the general formula (4) and a compound having two hydroxyl groups within the molecule thereof, the ratio of the compound represented by the general formula (4) may preferably be from 50 to 600 mol %, more preferably from 80 to 300 mol %, still more preferably from 100 to 250 mol %, based on the compound having two hydroxyl groups within the molecule thereof.

In the etherification reaction according to the present invention (V), the method of mixing the compound represented by the general formula (4), the compound represented by the general formula (5) and the catalyst comprising a divalent copper compound is not particularly limited, and these components may be mixed by any of mixing methods. Specifically, the compound represented by the general formula (4), the compound represented by the general formula (5) and the catalyst comprising a divalent copper compound can be mixed by the same method as in the present invention (IV).

In the process for producing an ether compound according to the present invention (V), the catalyst comprising a divalent copper compound can be separated from the reaction mixture by a conventionally known method, similarly to the present invention (IV). Of course, the catalyst comprising a divalent copper compound which has been separated and recovered from the reaction mixture can be again used in the etherification reaction between the compound represented by the general formula (4) and the compound represented by the general formula (5).

The reaction temperature in the etherification reaction according to the present invention (V) is not particularly limited, and the etherification reaction may be conducted at any reaction temperature. The preferred reaction temperature in the present invention (V) is the same as that in the present invention (IV) and may somewhat vary depending on the boiling point under atmospheric pressure of the compound represented by the general formula (4) to be used for the reaction. When the boiling point under atmospheric pressure of the compound represented by the general formula (4) is from 50 to 200° C., the reaction temperature may preferably be from 30 to 250° C., more preferably from 50 to 200° C. When the compound represented by the general formula (4) has a boiling point exceeding 200° C. under atmospheric pressure, the reaction temperature may preferably be from 80 to 300° C., more preferably from 100 to 250° C. Along with the change in the reaction with the elapse of time, the etherification reaction can be of course conducted by changing the reaction temperature within the above-described range. If the reaction temperature is less than the preferred temperature, the etherification reaction may proceed at a low reaction rate and this is not practical. On the other hand, if the reaction temperature exceeds the preferred temperature, the amount of high molecular weight impurities produced as by-products may be increased and this is not preferred.

In the etherification reaction according to the present invention (V), the reaction pressure is not particularly limited and the etherification reaction may be conducted under any reaction pressure. Similarly to the present invention (IV), the reaction pressure may be, for example, preferably from 0 to 4.0 MPaG (gauge pressure), more preferably from 0 to 3.0 MPaG. In a case where the etherification reaction according to the present invention is conducted by using a closed-type reactor, the composition of the constituent components in the reactor may change with the progress of the reaction and therefore, the reaction pressure does not show a constant value and may take any value in a predetermined range. Even in such a case, an ether compound can be obtained without any problem.

Also in the etherification reaction according to the present invention (V), when water is previously allowed to be present in the reaction system, an ether compound can be produced with high efficiency and high selectivity, similarly to the present invention (IV). The concentration of water allowed to be present is not particularly limited but the water concentration may preferably be from 0.1 to 50% by mass, more preferably from 0.5 to 40% by mass, still more preferably from 1.0 to 30% by mass, based on the ether compound which is a raw material compound. In this case, the water previously allowed to be present of course includes water which is present as a hydrate (for example, hydrate of a divalent copper compound). The compound represented by the general formula (4) and the compound represented by the general formula (5), which are used in the process for producing an ether compound with high efficiency and high selectivity by previously allowing water to be present within the reaction system, are not particularly limited. Preferred examples of the compound represented by the general formula (5) may include: compounds having a high solubility in water, more specifically, methanol, ethanol, propanol, allyl alcohol and phenol.

In the present invention (V), the crude product which has been obtained by separating the catalyst comprising a divalent copper compound from the mixture after reaction may be purified by a conventionally known method, whereby an ether compound having a high purity can be obtained. The method of purification is not particularly limited. For example, an ether compound having a high purity can be obtained by subjecting a reaction mixture free of the catalyst comprising a divalent copper compound to at least one unit separating operation selected from the group consisting of: distillation, extraction, liquid-liquid separation, membrane separation and crystallization. Even if the catalyst comprising a divalent copper compound remains in the reaction mixture, an ether compound can be of course easily purified.

(Present Invention (VI))

The present invention (VI) is described below. The present invention (VI) is an ether compound produced by the process for producing an ether compound according to the present invention (IV) or (V).

The reaction mixture containing an ether compound, which has been obtained by the process for producing an ether compound according to the present invention (IV) or (V), generally has a low by-product content because of the high-selectivity reaction. Therefore, the purification of an intended product can be attained by a simple treatment and in turn, a high-purity ether compound can be obtained. The structure of the ether compound according to the present invention (VI) is not particularly limited. The structure of the ether compound may include those as described in the description of the present invention (IV) or the present invention (V).

The present invention is described in more detail below by referring to Examples, but these Examples are only to show the outline or preferred embodiments of the invention and the present invention is not limited to these Examples.

### EXAMPLES

### Description of Terms in Examples and Comparative Examples

Conversion of Raw Material Compound

Conversion of Ally Alcohol in Etherification Reaction of Allyl Alcohol

This conversion shows a molar ratio of allyl alcohol which has been consumed during the etherification reaction, to ally alcohol charged before the reaction. The conversion was calculated according to the following formula:

$$\begin{matrix}\begin{matrix}Conversion ( \% ) \\ of allyl alcohol\end{matrix} = 100 \times \{ allyl alcohol consumed ( mol ) \} / \\ \{ amount of allyl alcohol charged before \\ reaction ( mol ) \}\end{matrix}$$

Conversion of n-Butyl Alcohol in Etherification Reaction Between n-Butyl Alcohol and Allyl Alcohol

This conversion shows a molar ratio of n-butyl alcohol which has been consumed during the etherification reaction, to n-butyl alcohol charged before the reaction. The conversion was calculated according to the following formula:

$$\begin{matrix}\begin{matrix}Conversion ( \% ) \\ of n \text{-} butyl alcohol\end{matrix} = 100 \times \{ n \text{-} butyl alcohol consumed ( mol ) \} / \\ \{ amount of n \text{-} butyl alcohol charged before \\ reaction ( mol ) \}\end{matrix}$$

Conversion of n-Butyl Alcohol in Etherification Reaction Between n-Butyl Alcohol and Diallyl Ether

This conversion shows a molar ratio of n-butyl alcohol which has been consumed during the etherification reaction, to n-butyl alcohol charged before the reaction. The conversion was calculated according to the following formula:
Conversion (%) of n-butyl alcohol=100×{n-butyl alcohol consumed (mol)}/{amount of n-butyl alcohol charged before reaction (mol)}
Selectivity of Unsaturated Ether Compound
Selectivity of Diallyl Ether in Etherification Reaction of Allyl Alcohol

This selectivity shows a ratio of allyl alcohol which has been consumed in the etherification reaction, to diallyl ether produced. The selectivity was calculated according to the following formula. At this time, the allyl alcohol which has been consumed in the reaction was calculated from the difference between the amount of the allyl alcohol before the reaction, and the amount thereof after the reaction.

$$\begin{matrix}\begin{matrix}Selectivity ( \% ) \\ of diallyl ether\end{matrix} = 2 \times 100 \times \{ diallyl ether produced ( mol ) \} / \\ \{ allyl alcohol before reaction ( mol ) - \\ allyl alcohol after reaction ( mol ) \}\end{matrix}$$

Selectivity of Allyl Butyl Ether in Etherification Reaction Between n-Butyl Alcohol and Allyl Alcohol

This selectivity shows a ratio of n-butyl alcohol which has been consumed in the etherification reaction, to allyl butyl ether produced. The selectivity was calculated according to the following formula. At this time, the n-butyl alcohol which has been consumed in the reaction was calculated from the difference between the amount of the n-butyl alcohol before the reaction, and the amount thereof after the reaction.

$$\begin{matrix}\begin{matrix}Selectivity ( \% ) of \\ allyl butyl ether\end{matrix} = 100 \times \{ allyl butyl ether produced ( mol ) \} / \\ \{ n \text{-} butyl alcohol before reaction ( mol ) - \\ n \text{-} butyl alcohol after reaction ( mol ) \}\end{matrix}$$

Selectivity of Allyl Butyl Ether in Etherification Reaction Between n-Butyl Alcohol and Diallyl Ether

This selectivity shows a ratio of n-butyl alcohol which has been consumed in the etherification reaction, to allyl butyl ether produced and was calculated according to the following formula. At this time, the n-butyl alcohol which has been consumed in the reaction was calculated from the difference between the amount of the n-butyl alcohol before the reaction, and the amount thereof after the reaction.

$$\begin{matrix}\begin{matrix}Selectivity ( \% ) of \\ allyl butyl ether\end{matrix} = 100 \times \{ allyl butyl ether produced ( mol ) \} / \\ \{ n \text{-} butyl alcohol before reaction ( mol ) - \\ n \text{-} butyl alcohol after reaction ( mol ) \}\end{matrix}$$

### Analyzing Apparatus Used in Examples and Comparative Examples

Analysis of Organic Compound Concentration in Filtrate of Reaction Mixture

The concentration was measured by the following gas chromatograph under the following analyzing conditions.

The analysis was conducted by using an internal standard method, where 1 g of 1,4-dioxane (mfd. by Wako Pure Chemical Industries, Ltd., guaranteed reagent) as the internal standard was added to 10 g of the reaction mixture to prepare an analyte solution, and 0.4 μl of the analyte solution was injected into the gas chromatograph.
Gas chromatograph:
   GC-14B, mfd. by Shimadzu Corporation
Column:
   capillary column TC-WAX (length: 30 m, internal diameter: 0.25 mm, wall thickness: 0.25 μm)
Carrier gas:
   nitrogen (split ratio: 20, column flow rate: 2 ml/min)
Temperature conditions:

The temperatures of detector and vaporizing chamber were 200° C.

The temperature of column was kept at 50° C. for 5 minutes from the start of analysis, then was elevated to 150° C. at a temperature-rising rate of 10° C./min, kept at 150° C. for 10 minutes, thereafter elevated to 200° C. at a temperature-rising rate of 10° C./min and kept at that temperature for 25 minutes.
Detector:
   FID (H₂ pressure: 70 kPa, air pressure: 100 kPa)

### Example 1

### Etherification Reaction of Allyl Alcohol

An etherification reaction of allyl alcohol was conducted by using a 1 L-volume glass autoclave reaction apparatus (HYPERGLASSTER TEM-V1000N, mfd. by Taiatsu Techno Corporation) equipped with a stirring shaft coated with Teflon (registered trademark) and a thermocouple for measuring the internal temperature. In the reactor, 350 g (6.03 mol) of allyl alcohol and 10.28 g (60.3 mmol) of copper(II) chloride dihydrate (mfd. by Wako Pure Chemical industries, Ltd., guaranteed reagent) were added. The reactor was tightly closed and no leakage within the system was confirmed by conducting an air-tightness test using nitrogen. Thereafter, the pressure in the system was returned to atmospheric pressure, stirring was conducted at a rotation number of 350 rpm, and the power of a heater outside the reactor was turned on to start heating. The time point at which the temperature inside the reactor reached 150° C., was assigned as the reaction starting time, and the reaction was conducted until 3.0 hours passed from the reaction starting time. After the reaction was continued for 3.0 hours, the heating by the external heater was stopped and the system was externally cooled by using a dry nitrogen gas. When the temperature inside the reactor was lowered to 35° C. or less, the reaction mixture inside the reactor was taken out from the reactor. Since two phases of an organic phase and an aqueous phase were formed in the reaction mixture, the reaction mixture taken out was separated into an organic phase and an aqueous phase by using a separating funnel, and the weight of each phase was measured. Subsequently, each of the aqueous phase and the organic phase was analyzed by gas chromatography, and the reaction results were finally calculated from the respective analytical values and weights. The conversion of allyl alcohol was 84.7%, and the selectivity of diallyl ether based on allyl alcohol was 97.1%. The reaction results are shown in the following Table 1.

TABLE 1

| | Catalyst | | | | Reaction | Conversion | Selectivity |
|---|---|---|---|---|---|---|---|
| | | Amount Used | | Reaction | Time | of Allyl | of Diallyl |
| | Kind | (mmol ) | Co-catalyst | Temp. (° C.) | (hr) | Alcohol*¹ (%) | Ether*² (%) |
| Example 1 | CuCl₂.2H₂O | 60.3 | None | 150 | 3.0 | 84.7 | 97.1 |
| Example 2 | CuCl₂.2H₂O | 60.3 | None | 155 | 2.0 | 83.1 | 98.0 |
| Example 3 | CuCl₂.2H₂O | 180.9 | None | 150 | 1.0 | 86.2 | 94.3 |
| Comp. | CuCl | 603.0 | NH₄Cl (301.5 mmol) | 150 | 1.0 | 73.2 | 95.2 |
| Example 1 | | | | | | | |
| Comp. | CuCl | 603.0 | NH₄Cl (603.0 mmol) | 150 | 3.0 | 63.6 | 91.5 |
| Example 2 | | | | | | | |
| Comp. | CuCl | 60.3 | NH₄Cl (30.2 mmol) | 150 | 3.0 | 12.0 | 95.7 |
| Example 3 | | | | | | | |

*¹Conversion of allyl alcohol: {allyl alcohol consumed (mol)/raw material allyl alcohol (mol)} × 100 (%)
*²Selectivity of diallyl ether: {diallyl ether produced (mol)/allyl alcohol consumed (mol)/2} × 100 (mol %)

### Example 2

### Etherification Reaction of Allyl Alcohol

The procedure was repeated in the same manner as in Example 1, except that the etherification reaction was conducted at a reaction temperature of 155° C. and the reaction time was changed to 2.0 hours. The reaction results are shown in the above Table 1.

### Example 3

### Etherification Reaction of Allyl Alcohol

The procedure was repeated in the same manner as in Example 1, except that the copper(II) chloride dihydrate was used in an amount of 30.84 g (180.9 mmol) and the reaction time was changed to 1.0 hour. The reaction results are shown in the above Table 1.

### Comparative Example 1

### Etherification Reaction of Allyl Alcohol

The procedure was repeated in the same manner as in Example 1, except that 54.3 g (603 mmol) of copper (I) chloride and 16.1 g (302 mmol) of ammonium chloride were used instead of the copper(II) chloride dihydrate, and the reaction time of the etherification reaction was changed to 1.0 hour. The reaction results are shown in the above Table 1.

### Comparative Example 2

### Etherification Reaction of Allyl Alcohol

The procedure was repeated in the same manner as in Example 1, except that 54.3 g (603 mmol) of copper (I) chloride and 32.2 g (603 mmol) of ammonium chloride were used instead of the copper(II) chloride dihydrate. The reaction results are shown in the above Table 1.

### Comparative Example 3

### Etherification Reaction of Allyl Alcohol

The procedure was repeated in the same manner as in Example 1, except that 5.43 g (60.3 mmol) of copper (I) chloride and 1.61 g (30.2 mmol) of ammonium chloride were used instead of the copper(II) chloride dihydrate. The reaction results are shown in the above Table 1.

### Example 4

### Etherification Reaction of Allyl Alcohol

An etherification reaction of allyl alcohol was conducted by using an autoclave reaction apparatus (portable reactor Model TPR-1, mfd. by Taiatsu Techno Corporation, material of reactor: sus316) equipped with a 120 ml-volume inner cylinder made of Teflon (registered trademark).

In a reactor with a magnetic stirrer, 30 g of an aqueous 70 mass %-allyl alcohol solution (allyl alcohol: 27 g (517 mmol), distilled water: 3.0 g (166 mmol)) and 0.176 g (1.03 mmol) of copper(II) chloride dihydrate (mfd. by Wako Pure Chemical Industries, Ltd., guaranteed reagent) were added. The reactor was tightly closed and no leakage within the system was confirmed by conducting an air-tightness test using nitrogen. Thereafter, the pressure in the system was returned to atmospheric pressure, stirring was conducted at a rotation number of 350 rpm, and heating was started by fixing an electric heater to the outside of the reactor. The time point at which the temperature inside the reactor reached 100° C. (predetermined temperature), was assigned as the reaction starting time, and the stirring was continued for 15 minutes from the reaction starting time. After 15 minutes passed, the external electric heater was removed from the reactor and the reactor was externally cooled by using an ice bath to stop the reaction. After the confirmation that the temperature inside the reactor was lowered to 35° C. or less, all of the contents were taken out from the reactor. Methanol was added to the reaction mixture which had been taken out from the reactor, and the total weight thereof was measured and then the reaction mixture was analyzed by gas chromatography. From the analytical values and weight, the reaction results were finally calculated. The conversion of allyl alcohol was 5.4% and the selectivity of diallyl ether based on allyl alcohol was 98.3%.

### Comparative Example 4

### Etherification Reaction of Allyl Alcohol

The etherification reaction of allyl alcohol was conducted in the same manner as in Example 4, except that 30 g (517 mmol) of 100 mass %-allyl alcohol was used instead of 30 g of an aqueous 70 mass %-allyl alcohol solution (allyl alcohol: 27 g (465 mmol), distilled water: 3.0 g (166 mmol)). The conversion of allyl alcohol was 2.5% and the selectivity of diallyl ether based on allyl alcohol was 91.2%.

### Example 5

### Etherification Reaction Between n-Butyl Alcohol and Allyl Alcohol

Etherification reaction of allyl alcohol was conducted by using an autoclave reaction apparatus (portable reactor Model TPR-1, mfd. by Taiatsu Techno Corporation, material of reactor: sus316) equipped with a 120 ml-volume inner cylinder made of Teflon (registered trademark).

In a reactor with a magnetic stirrer, 30 g (517 mmol) of allyl alcohol, 7.66 g (103 mmol) of n-butyl alcohol and 0.881 g (5.17 mmol) of copper(II) chloride dihydrate (mfd. by Wako Pure Chemical Industries, Ltd., guaranteed reagent) were added. The reactor was tightly closed and no leakage within the system was confirmed by conducting an air-tightness test using nitrogen. Thereafter, the pressure in the system was returned to atmospheric pressure, stirring was conducted at a rotation number of 350 rpm, and heating was started by fixing an electric heater to the outside of the reactor. The time point at which the temperature inside the reactor reached 155° C. (predetermined temperature), was assigned as the reaction starting time, and the stirring was continued for 1.0 hour from the reaction starting time. After 1.0 hour passed, the external electric heater was removed from the reactor and the reactor was externally cooled by using an ice bath to stop the reaction. After the confirmation that the temperature inside the reactor was lowered to 35° C. or less, all of the contents were taken out from the reactor. The weight of the reaction mixture taken out from the reactor was measured, and the reaction mixture was analyzed by gas chromatography. From the analytical values and weight, the reaction results were finally calculated. The conversion of n-butyl alcohol was 60.7% and the selectivity of allyl butyl ether based on n-butyl alcohol was 89.1%.

### Comparative Example 5

### Etherification Reaction Between n-Butyl Alcohol and Allyl Alcohol

An etherification reaction was conducted in the same manner as in Example 5, except that 1.16 g (5.17 mmol) of palladium acetate and 5.42 g (20.68 mmol) of triphenylphosphine were used instead of using 0.176 g (1.03 mmol) of copper(II) chloride dihydrate (mfd. by Wako Pure Chemical Industries, Ltd., guaranteed reagent). The conversion of n-butyl alcohol was 50.9% and the selectivity of allyl butyl ether based on n-butyl alcohol was 86.7%.

### Example 6

### Etherification Reactizon Between n-Butyl Alcohol and Diallyl Ether

Etherification reaction of allyl alcohol was conducted by using an autoclave reaction apparatus (portable reactor Model TPR-1, mfd. by Taiatsu Techno Corporation, material of reactor: sus316) equipped with a 120 ml-volume inner cylinder made of Teflon (registered trademark).

In a reactor with a magnetic stirrer, 30 g (306 mmol) of diallyl ether, 2.22 g (30 mmol) of n-butyl alcohol and 0.256 g (1.5 mmol) of copper(II) chloride dihydrate (mfd. by Wako Pure Chemical Industries, Ltd., guaranteed reagent) were added. The reactor was tightly closed and no leakage within the system was confirmed by conducting an air-tightness test using nitrogen. Thereafter, the pressure in the system was returned to atmospheric pressure, stirring was conducted at a rotation number of 350 rpm, and heating was started by fixing an electric heater to the outside of the reactor. The time point at which the temperature inside the reactor reached 155° C. (predetermined temperature), was assigned as the reaction starting time, and the stirring was continued for 1.0 hour from the reaction starting time. After 1.0 hour passed, the external electric heater was removed from the reactor and the reactor was externally cooled by using an ice bath to stop the reaction. After the confirmation that the temperature inside the reactor was lowered to 35° C. or less, all of the contents were taken out from the reactor. The weight of the reaction mixture taken out from the reactor was measured, and the reaction mixture was analyzed by gas chromatography. From the analytical values and weight, the reaction results were finally calculated. The conversion of n-butyl alcohol was 8.9% and the selectivity of allyl butyl ether based on n-butyl alcohol was 22.5%.

### INDUSTRIAL APPLICABILITY

As described above, it is clear that the catalyst comprising a divalent copper compound according to the present invention is a very useful catalyst in the process for producing an ether compound by an etherification reaction between an alcohol compound and a compound having at least one hydroxyl group within the molecule thereof, or by an etherification reaction between an ether compound and a compound having at least one hydroxyl group within the molecule thereof, as compared with conventionally known catalysts for the production of an ether compound. It is also clear that the process for producing an ether compound according to the present invention can provides an ether compound with good efficiency and high selectivity.

1. A catalyst for producing an ether compound, comprising at least one copper compound selected from the group consisting of: copper(II) sulfate, copper(II) ammonium chloride, copper(II) carbonate, copper(II) diphosphate, copper(II) formate, copper(II) gluconate, copper(II) hydroxide, copper(II) nitrate, copper(II) oleate, copper(II) oxalate, copper(II) sulfide, copper(II) phthalate, copper(II) phthalocyanine, copper(II) potassium chloride, copper(II) terephthalate, copper(II) thiocyanate, copper(II) chloride, copper(II) bromide, copper(II) fluoride, copper(II) iodide, copper(II) oxide, copper(II) acetate, bis(acetylacetonato)-copper(II); and hydrates of these compounds.

2. The catalyst for producing an ether compound according to claim 1, which is a catalyst usable in the production of an ether compound represented by the general formula (3) from an alcohol compound represented by the general formula (1) and a compound represented by the general formula (2):
Formula (1): 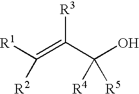
(wherein R¹, R², R³, R⁴ and R⁵ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R⁶—OH Formula (2):
(wherein R⁶ represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
Formula (3): 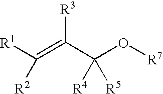
(wherein R¹, R², R³, R⁴, R⁵ and R⁷ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

3. The catalyst for producing an ether compound according to claim 1, which is a catalyst usable in the production of an ether compound represented by the general formula (6) from an ether compound represented by the general formula (4) and a compound represented by the general formula (5):
R⁸—O—R⁹ Formula (4):
(wherein R⁸ and R⁹ each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹⁰—OH Formula (5):
(wherein R¹⁰ represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹¹—O—R¹² Formula (6):
(wherein R¹¹ and R¹² each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

4. A process for producing an ether compound represented by the general formula (3), comprising conducting an etherification reaction between a compound represented by the general formula (1) and a compound represented by the general formula (2) in the presence of the catalyst for producing an ether compound according to claim 2:
Formula (1): 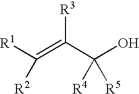
(wherein R¹, R², R³, R⁴ and R⁵ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R⁶—OH Formula (2):
(wherein R⁶ represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
Formula (3): 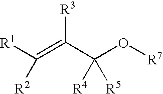
(wherein R¹, R², R³, R⁴, R⁵ and R⁷ each independently represents at least one member selected from hydrogen, an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

5. The process for producing an ether compound according to claim 4, wherein the alcohol compound represented by the general formula (1) is a compound having from 2 to 20 carbon atoms.

6. The process for producing an ether compound according to claim 4 or 5, wherein the alcohol compound represented by the general formula (1) is at least one compound selected from the group consisting of: allyl alcohol, 2-methyl-2-propen-1-ol, 3-buten-2-ol, 2,3-dimethyl-3-buten-2-ol, 3-methyl-3-buten-2-ol, 2-buten-1-ol, 2-methyl-3-buten-1-ol and 3-penten-2-ol.

7. The process for producing an ether compound according to any one of claims 4 to 6, wherein the compound represented by the general formula (2) is at least one compound selected from the group consisting of: alcohol compounds, phenol compounds, polycondensation reaction products from a phenol compound and an aldehyde compound, and polyaddition reaction products from a phenol compound and an unsaturated hydrocarbon compound.

8. The process for producing an ether compound according to claim 7, wherein the alcohol compound is at least one compound selected from the group consisting of: vinyl alcohol, 2-methylvinyl alcohol, allyl alcohol, 2-methyl-2-propen-1-ol, 3-buten-2-ol, 2,3-dimethyl-3-buten-2-ol, 3-methyl-3-buten-2-ol, 2-buten-1-ol, 2-methyl-3-buten-1-ol, 3-penten-2-ol, ethylene glycol, ethylene glycol mono-substituted product, 1,2-propanediol, 1,2-propanediol mono-substituted product, 1,3-propanediol, 1,3-propanediol mono-substituted product, 1,2-butanediol, 1,2-butanediol mono-substituted product, 1,3-butanediol, 1,3-butanediol mono-substituted product, 1,4-butanediol, 1,4-butanediol mono-substituted product, trimethylol propane, trimethylol propane mono-substituted product, trimethylol propane di-substitution product, pentaerythritol, pentaerythritol mono-substituted product, pentaerythritol di-substitution product, pentaerythritol tri-substituted product, methanol, ethanol, 1-propanol, 2-propanol, 1-butanol, 2-butanol, tert-butanol and benzyl alcohol.

9. The process for producing an ether compound according to claim 7 or 8, wherein the phenol compound is at least one compound selected from the group consisting of: unsubstituted phenol, mono-substituted phenol compounds, di-substituted phenol compounds, tri-substituted phenol compounds, dihydric phenol compounds and naphthol compounds.

10. The process for producing an ether compound according to any one of claims 4 to 9, wherein the alcohol compound represented by the general formula (1) is allyl alcohol.

11. A process for producing an ether compound represented by the general formula (6), comprising conducting an etherification reaction between a compound represented by the general formula (4) and a compound represented by the general formula (5) in the presence of the catalyst for producing an ether compound according to claim 3:
R⁸—O—R⁹ Formula (4):
(wherein R⁸ and R⁹ each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹⁰—OH Formula (5):
(wherein R¹⁰ represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms);
R¹¹—O—R¹² Formula (6):
(wherein R¹¹ and R¹² each independently represents at least one member selected from an alkyl group having from 1 to 20 carbon atoms, an alkenyl group having from 2 to 20 carbon atoms, an alkynyl group having from 2 to 20 carbon atoms and an aryl group having from 6 to 20 carbon atoms).

12. The process for producing an ether compound according to claim 11, wherein the compound represented by the general formula (4) is at least one compound selected from the group consisting of: allyl ether compounds, vinyl ether compounds and propenyl ether compounds, and the compound represented by the general formula (5) is at least one compound selected from the group consisting of: alcohol compounds, phenol compounds, polycondensation reaction products from a phenol compounds and an aldehyde compound, and polyaddition reaction products from a phenol compound and an unsaturated hydrocarbon compound.

13. The process for producing an ether compound according to claim 11 or 12, wherein the ether compound represented by the general formula (4) is diallyl ether.

14. The process for producing an ether compound according to any one of claims 4 to 13, wherein water is present in the reaction system.

15. The process for producing an ether compound according to claim 14, wherein the amount of water present in the reaction system is from 0.01 to 40% by mass.

16. An ether compound produced by the process for producing an ether compound according to any one of claims 4 to 15.
